(19) United States
(12) Patent Application Publication
(10) Pub. No.: US 2026/0315654 A1
(43) Pub. Date: Oct. 15, 2026

(54) CORROSION-RESISTANT MAGNESIUM ALLOY COMPOSITE COATING FOR PROMOTING BONE HEALING, PREPARATION METHOD THEREFOR AND MEDICAL MAGNESIUM ALLOY

(71) Applicant: Hua Rong Ke Chuang Biotechnology(Tian Jin) Co.,Ltd, Tianjin (CN)

(72) Inventors: Lina Liu, Tianjin (CN); Zhongying Zhang, Tianjin (CN)

(21) Appl. No.: 19/643,694

(22) Filed: Apr. 9, 2026

(30) Foreign Application Priority Data
Apr. 10, 2025 (CN) 202510443615.X

(51) Int. Cl. A61L 27/30; A61L 27/32; A61L 27/34

(52) U.S. Cl. CPC A61L 27/306; A61L 27/32; A61L 27/34; A61L 2400/18; A61L 2420/02; A61L 2430/02

(57) ABSTRACT

Provided are a corrosion-resistant magnesium alloy composite coating for promoting bone healing, a preparation method therefor and a medical magnesium alloy, where the magnesium alloy composite coating includes a base coating and a surface coating which are sequentially arranged outward from the surface of a magnesium alloy; the base coating includes microporous channels, and the component of the base coating includes magnesium hydroxide and/or magnesium oxide; the surface coating includes a polymer material, the polymer material includes polylactic acid or a mixture of polylactic acid and polycaprolactone, and the surface coating fills the microporous channels of the base coating and covers the surface of the base coating. By bonding the base coating formed through a chemical conversion method to the surface coating formed by a polymer material in an interdigitation manner, the magnesium alloy composite coating provided by the present disclosure has good firmness, strength and toughness, which improves the corrosion resistance of the magnesium alloy, and meets the need of promoting bone healing.

This application claims priority to Chinese Patent Application No. 202510443615.X filed with CNIPA on Apr. 10, 2025, the disclosure of which is incorporated herein by reference in its entirety.

### TECHNICAL FIELD

The present disclosure relates to the technical field of biomedical materials and specifically, to a corrosion-resistant magnesium alloy composite coating for promoting bone healing, a preparation method therefor and a medical magnesium alloy.

### BACKGROUND

Magnesium is an essential element in the human body. Magnesium alloys exhibit favorable biocompatibility and metabolic mechanisms and have broad application prospects in the medical field. However, inadequate corrosion resistance and excessively rapid degradation rates of magnesium alloys limit their widespread application.

In existing research, the corrosion resistance of magnesium alloys has been improved through approaches such as alloying treatment, impurity content control, and processing optimization. Nevertheless, the potential for further enhancement remains limited. In many application scenarios, magnesium alloys still cannot be deployed without modification. Consequently, surface modification of magnesium alloys is imperative, which can greatly enhance the corrosion resistance of magnesium alloys and is the primary research direction for magnesium alloy applications. Numerous surface modification techniques exist for magnesium alloys, with key considerations including biocompatibility, degradation capabilities, and the effectiveness of degradation inhibition of magnesium alloys.

For example, CN118105548A discloses a medical degradable magnesium alloy coating and a preparation method therefor. The medical degradable magnesium alloy coating includes a magnesium hydroxide coating with a thickness of 15 μm to 20 μm or a magnesium hydroxide-calcium phosphate composite coating with a thickness of 50 μm to 150 μm, and the magnesium hydroxide-calcium phosphate composite coating includes a magnesium hydroxide layer and a scale-shaped calcium phosphate layer attached to the surface of the magnesium hydroxide layer. The method disclosed herein adopts a hydrothermal method to prepare the degradable magnesium alloy coating. Although this method moderately improves corrosion rates, the coating exhibits limited strength and toughness and is prone to damage, resulting in substrate exposure.

For magnesium alloys used in magnesium alloy bone plates and nails, if the bonding strength of the coating is insufficient, the high friction generated during the torsional screw of the bone nails may form microcracks on the magnesium alloy, exposing the substrate magnesium alloy and accelerating localized corrosion. As a result, the magnesium alloy coating is required to possess superior toughness and strength to prevent the coating from cracking or delamination under high shearing forces generated during the torsional screw of the bone nails, enabling the magnesium alloy to maintain good corrosion resistance throughout the service period and meet the needs of bone healing.

Therefore, providing a magnesium alloy coating capable of improving the bonding strength between the coating and the substrate, enhancing corrosion resistance and promoting bone healing, and a preparation method therefor is a technical problem to be solved in the art.

### SUMMARY

In view of deficiencies in the related art, the object of the present disclosure is to provide a corrosion-resistant magnesium alloy composite coating for promoting bone healing, a preparation method therefor and a medical magnesium alloy. Compared to the related art, by bonding a base coating formed through a chemical conversion method to a surface coating formed by a polymer material in an interdigitation manner, the magnesium alloy composite coating provided by the present disclosure has good firmness, strength and toughness, which improves the corrosion resistance of the magnesium alloy, and meets the need of promoting bone healing.

To achieve the object, the present disclosure adopts the following technical solutions.

In a first aspect, the present disclosure provides a corrosion-resistant magnesium alloy composite coating for promoting bone healing. The magnesium alloy composite coating includes a base coating and a surface coating which are sequentially arranged outward from the surface of the magnesium alloy.

The base coating includes microporous channels, and a component of the base coating includes magnesium hydroxide and/or magnesium oxide.

The surface coating includes a polymer material, and the polymer material includes polylactic acid or a mixture of polylactic acid and polycaprolactone; the surface coating fills the microporous channels of the base coating and covers the surface of the base coating.

In the composite coating provided by the present disclosure, the base coating formed on the surface of the magnesium alloy through a hydrothermal reaction exhibits a highly compact structure and superior bonding strength to the substrate magnesium alloy. The base coating includes magnesium hydroxide and/or magnesium oxide as its main components and does not include other components or any impurities, and magnesium hydroxide and/or magnesium oxide, as intermediate products during the degradation of the magnesium alloy, enable the base coating to have excellent biocompatibility. The main component of the surface coating is polylactic acid or a mixture of polylactic acid and polycaprolactone. By using a combination of polycaprolactone and polylactic acid, the problem of insufficient toughness caused by polylactic acid can be mitigated while ensuring the strength of polylactic acid, thereby effectively improving the toughness of the polymer surface coating. When the magnesium alloy product needs to be reshaped, the polymer surface coating is prevented from cracking due to deformation. The composite coating achieves balanced strength-toughness properties and has good application prospects, especially in the field of magnesium alloys used in magnesium alloy bone plates and nails.

It is to be noted that, in one aspect, the base coating has higher surface roughness than the surface of the magnesium alloy and provides better interface for the adhesion of the polymer surface coating, thereby enhancing the bonding strength between the polymer surface coating and the magnesium alloy product and improving corrosion resistance; on another aspect, the microporous channels on the base coating enable the surface coating and the base coating to interdigite each other, thereby improving the bonding force between the composite coating and the substrate, and maintaining the properties of resistance to damage and corrosion resistance under the action of external forces. In summary, the composite coating provided by the present disclosure has excellent biocompatibility, can be completely degradable, exhibits great strength and toughness, and can persistently and effectively inhibit the degradation of the magnesium alloy, thereby improving the corrosion resistance of the magnesium alloy and promoting rapid bone healing.

Preferably, nano-hydroxyapatite is distributed within the polymer material. A particle size of the nano-hydroxyapatite is 20 nm to 60 nm, for example, 20 nm, 25 nm, 30 nm, 35 nm, 40 nm, 45 nm, 50 nm, 55 nm, or 60 nm. However, the particle size of the nano-hydroxyapatite is not limited to the numerical values listed above, and other numerical values unlisted within the numerical range are also applicable.

It is to be noted that by preferably adding nano-hydroxyapatite to the polymer material in the surface coating of the present disclosure, nano-hydroxyapatite can be continuously released during the degradation of the coating, thereby improving the local microenvironment, enhancing adhesion, proliferation, and differentiation of osteocytes, and accelerating bone tissue regeneration. In addition, the alkalinity of nano-hydroxyapatite can neutralize the local acidic environment generated by the degradation of polylactic acid, thereby reducing inflammatory responses. In the present disclosure, by preferably controlling the particle size of the nano-hydroxyapatite to be within a specific range, the nano-hydroxyapatite can be uniformly distributed in the polylactic acid coating. When the particle size of the nano-hydroxyapatite is excessively large, the nano-hydroxyapatite is prone to sedimentation in the solution and thus clogs the nozzle during ultrasonic spraying, leading to overly high content of polylactic acid coating per unit, and the coating cannot be completely coated by polylactic acid, thereby affecting the coating quality and compromising corrosion resistance. When the particle size of the nano-hydroxyapatite is too small, the nanoparticles has high specific surface area and surface energy and are prone to agglomeration to form crack sources, and in addition, the nanoparticles are more easily to be carried by airflow and lost during ultrasonic spraying, thereby resulting in an increased porosity and weakened interface bonding strength, degrading the uniformity and mechanical properties of the polylactic acid coating, and compromising the coating quality and corrosion resistance.

Preferably, a porosity of the base coating is 30% to 60%, for example, 30%, 35%, 40%, 45%, 55%, or 60%. However, the porosity of the base coating is not limited to the numerical values listed above, and other numerical values unlisted within the numerical range are also applicable. A pore diameter of the microporous channels is 20 μm to 120 μm, for example, 20 μm, 40 μm, 60 μm, 80 μm, 100 μm, or 120 μm. The pore diameter of the microporous channels is not limited to the numerical values listed above, and other numerical values unlisted within the numerical range are also applicable.

In the present disclosure, by forming pores on the base coating, an interdigitating mechanical interlock structure can be formed without completely damaging the magnesium alloy substrate, thereby enhancing the bonding strength of the polymer coating on the surface. When subjected to external forces, the coating is less prone to damage, thereby improving corrosion resistance and forming robust composite structures among the surface coating, the base coating, and the substrate. In the present disclosure, by preferably controlling the porosity and the pore diameter of the base coating within specific ranges, the values of the porosity and the pore diameter should not be excessively large, thereby meeting the primary objective to match the porosity and the pore diameter of the base coating with the molecular weight and the structure of polylactic acid. Therefore, an interdigitation structure is more likely to be formed between polylactic acid and the micropores during ultrasonic spraying to achieve full-surface engagement of the magnesium alloy product, thereby enhancing the bonding strength and avoiding localized accumulation of polylactic acid.

Preferably, the thickness of the base coating is 8 μm to 12 μm, for example, 8 μm, 9 μm, 10 μm, 11 μm, or 12 μm. The thickness of the base coating is not limited to the numerical values listed above, and other numerical values unlisted within the numerical range are also applicable.

Preferably, the thickness of the surface coating is 15 μm to 30 μm, for example, 15 μm, 16 μm, 18 μm, 20 μm, 22 μm, 24 μm, 26 μm, 28 μm, or 30 μm. The thickness of the surface coating is not limited to the numerical values listed above, and other numerical values unlisted within the numerical range are also applicable.

In the present disclosure, the thickness of the surface coating is preferably controlled within a specific range, because the coating thickness significantly affects the degradation cycle and the bonding strength. When the coating is excessively thick, corrosion resistance can be improved, and theoretically, the degradation cycle can be extended. In practice, when the coating thickness exceeds a critical threshold, the internal stress within the coating increases, and the bonding strength decreases. Consequently, when subjected to external forces, the coating becomes prone to cracking, thereby resulting in reduced corrosion resistance and a shortened degradation cycle. When the coating is excessively thin, corrosion resistance is reduced, the degradation cycle is shortened, and the coating fails to provide sufficient protective effects for the magnesium alloy substrate.

In a second aspect, the present disclosure provides a method for preparing the corrosion-resistant magnesium alloy composite coating for promoting bone healing described in the first aspect. The preparation method includes the following steps.
(1) Subjecting water vapor to a hydrothermal reaction with a magnesium alloy substrate to obtain a substrate containing a base coating.
(2) Immersing the substrate containing the base coating obtained in step (1) into a phosphate buffer solution for soaking to obtain a base coating containing microporous channels.
(3) Ultrasonic spraying the base coating obtained in step (2) by using a spraying solution containing a polymer material, and drying the base coating to complete the preparation of the magnesium alloy composite coating.

In the present disclosure, chemical conversion is carried out on a magnesium alloy used as the substrate through a hydrothermal reaction to form a dense yet rough base coating on the surface of the magnesium alloy. Subsequently, microporous channels are formed on the base coating by pore formation. A polymer material is then sprayed onto the surface of the base coating via a specifically selected ultrasonic spraying process to enable the surface coating and the base coating to form an interdigitation structure, and then a composite coating is obtained. The composite coating has excellent biocompatibility, able to completely degrade, exhibits great strength and toughness, and can persistently and effectively inhibit the degradation of the magnesium alloy, thereby improving the corrosion resistance of the magnesium alloy and promoting rapid bone healing.

It is to be noted that in the present disclosure, by specifically adopting the ultrasonic spraying process to prepare the surface coating, the surface coating is enabled to be matched with the roughness of the surface of the base coating and the microporous channels on the base coating. Through ultrasonic oscillation atomization (typically performed at 20° C. to 60° C.) of ultrasonic spraying, the polymer solution (or suspension) can be broken into droplets of 5 μm to 50 μm in a liquid or semi-solid state, and these droplets are directly deposited onto the base coating and inside the microporous channels, thereby forming a smooth, dense coating. Therefore, high-temperature damage is avoided, the molecular structure and biocompatibility of the polymer are preserved, the magnesium alloy is prevented from oxidation, and mechanical interlocking is achieved, thereby enhancing the bonding among the substrate, the base coating, and the surface coating. By adjusting the parameters of ultrasonic during ultrasonic spraying, the thickness of the surface coating can be precisely controlled, thereby meeting the mechanical requirements of different sites. In addition, nano-hydroxyapatite or other functional components may be added to the spraying solution adopted during ultrasonic spraying as needed for synchronous spraying, and the dispersion degree of the nano-hydroxyapatite is greater than 95%, thereby ensuring the formation of a uniform composite coating, avoiding issues such as decomposition or agglomeration of nano-hydroxyapatite encountered with other spraying techniques including plasma spraying in the related art, allowing the nano-hydroxyapatite to be gradually released to exert its efficacy, and enhancing the bone healing efficiency.

In the present disclosure, the method in step (1) for preparing a magnesium hydroxide and/or magnesium oxide coating through a hydrothermal reaction between water vapor and the magnesium alloy substrate is a conventional method in the art. For example, the preparation method may be performed as follows: the magnesium alloy substrate is suspended inside a reaction vessel containing purified water, the vessel is sealed, and then the hydrothermal reaction is carried out within the reaction vessel. The hydrothermal reaction is carried out at a temperature of 120° C. to 160° C., for example, 120° C., 130° C., 140° C., 150° C., or 160° C. The temperature for the hydrothermal reaction is not limited to the numerical values listed above, and other numerical values unlisted within the numerical range are also applicable. The hydrothermal reaction is carried out at a pressure of 1.0 MPa to 2.0 MPa, for example, 1.0 MPa, 1.5 MPa, or 2.0 MPa. The pressure for the hydrothermal reaction is not limited to the numerical values listed above, and other numerical values unlisted within the numerical range are also applicable. The hydrothermal reaction is carried out for 2 h to 6 h, for example, 2 h, 4 h, or 6 h. The time of the hydrothermal reaction is not limited to the numerical values listed above, and other numerical values unlisted within the numerical range are also applicable. In the present disclosure, the magnesium alloy substrate is generally subjected to pretreatment prior to the hydrothermal reaction. The pretreatment may be performed in a conventional method. The method for pretreatment typically includes the following steps: the magnesium alloy substrate is subjected to physical grinding, cleaning, and electrochemical polishing in sequence to remove surface contaminants; then, the magnesium alloy substrate is placed into a sealable reaction vessel, purified water is added to the bottom of the vessel, and the distance between the magnesium alloy substrate and the purified water is adjusted to be generally 30 mm to 50 mm (for example, the distance may be 30 mm, 40 mm, or 50 mm; the distance is not limited to the numerical values listed above, and other numerical values unlisted within the numerical range are also applicable); subsequently, the reaction vessel is sealed and placed into an equipment to commence the preparation of the base coating.

Preferably, the soaking in step (2) is carried out at a temperature of 60° C. to 80° C., for example, 60° C., 62° C., 64° C., 66° C., 68° C., 70° C., 72° C., 74° C., 76° C., 78° C., or 80° C. The temperature for the soaking is not limited to the numerical values listed above, and other numerical values unlisted within the numerical range are also applicable. The soaking is carried out for 5 min to 15 min, for example, 5 min, 8 min, 10 min, 12 min, 14 min, or 15 min. The time of the soaking is not limited to the numerical values listed above, and other numerical values unlisted within the numerical range are also applicable. A concentration of the phosphate buffer solution is 8 mmol/L to 15 mmol/L, for example, 8 mmol/L, 10 mmol/L, 12 mmol/L, 14 mmol/L, or 15 mmol/L. The concentration of the phosphate buffer solution is not limited to the numerical values listed above, and other numerical values unlisted within the numerical range are also applicable.

In the present disclosure, the phosphate buffer solution is a buffer solution commonly used in the art and may be prepared from a commercially available phosphate buffer solution (PBS).

In the present disclosure, by preferably controlling the parameters such as the concentration of the soaking solution, the time of the soaking, and the temperature for the soaking in the step (2), the porosity and the pore diameter of the microporous channels are controlled, and the base coating obtains some roughness to enable the porosity and the pore diameter to be matched with the molecular weight of polylactic acid used. Therefore, the base coating is more easily bonded to polylactic acid to achieve full-surface engagement of the magnesium alloy product, thereby enhancing the bonding strength and avoiding localized accumulation of polylactic acid. In the present disclosure, after the soaking, the substrate is generally taken out, washed with purified water and then air-dried.

Preferably, a solvent of the spraying solution in step (3) includes dichloromethane.

In the present disclosure, the spraying solution generally uses dichloromethane as the solvent, and polymer materials and functional components such as nano-hydroxyapatite are added to the solvent. The spraying solution may be prepared by using a conventional method in the art. The method for preparing the spraying solution generally includes the following steps: polylactic acid and polycaprolactone are added to dichloromethane, and then magnetically stirred at room temperature (20° C. to 25° C.) for 1 h to 2 h to fully dissolve polylactic acid and polycaprolactone with no suspended particles observed; nano-hydroxyapatite is added, and continuesly magnetically stirred for 15 min to 30 min to uniformly disperse nano-hydroxyapatite in the solution, thereby obtaining the spraying solution.

Preferably, a concentration of polylactic acid in the spraying solution in step (3) is 0.005 g/mL to 0.020 g/mL, for example, 0.005 g/mL, 0.006 g/mL, 0.008 g/mL, 0.010 g/mL, 0.012 g/mL, 0.014 g/mL, 0.016 g/mL, 0.018 g/mL, or 0.020 g/mL. The concentration of polylactic acid in the spraying solution is not limited to the numerical values listed above, and other numerical values unlisted within the numerical range are also applicable. A concentration of polycaprolactone in the spraying solution is 0.25 mg/mL to 1.50 mg/mL, for example, 0.25 mg/mL, 0.30 mg/mL, 0.40 mg/mL, 0.50 mg/mL, 0.60 mg/mL, 0.70 mg/mL, 0.80 mg/mL, 0.90 mg/mL, 1.00 mg/mL, 1.10 mg/mL, 1.20 mg/mL, 1.30 mg/mL, 1.40 mg/mL, or 1.50 mg/mL. The concentration of polycaprolactone in the spraying solution is not limited to the numerical values listed above, and other numerical values unlisted within the numerical range are also applicable.

Preferably, a concentration of nano-hydroxyapatite in the spraying solution in step (3) is 0.05 mg/mL to 0.50 mg/mL, for example, 0.05 mg/mL, 0.10 mg/mL, 0.15 mg/mL, 0.20 mg/mL, 0.25 mg/mL, 0.30 mg/mL, 0.35 mg/mL, 0.40 mg/mL, 0.45 mg/mL, or 0.50 mg/mL. The concentration of nano-hydroxyapatite in the spraying solution is not limited to the numerical values listed above, and other numerical values unlisted within the numerical range are also applicable.

In the present disclosure, by preferably controlling the concentration of nano-hydroxyapatite within a specific range, the dispersion, interfacial bonding, mechanical properties, and biological response of nano-hydroxyapatite can be further controlled, thereby enhancing the overall quality of the polylactic acid coating. When the concentration of nano-hydroxyapatite is excessively high, the solution viscosity tends to increase, and the spraying process then fails, and the coating shrinkage and cracking occur during drying; at the same time, the brittleness of the coating also increases, and fracture toughness is reduced, thereby compromising the coating quality. When the concentration of nano-hydroxyapatite is too low, the osteoblast activity cannot be effectively modulated, thereby resulting in reduced bone healing efficiency and impaired functional performance of the coating in bone healing applications.

Preferably, the weight-average molecular mass of polylactic acid in step (3) is 100000 Da to 200000 Da, for example, 100000 Da, 120000 Da, 140000 Da, 160000 Da, 180000 Da, or 200000 Da. The weight-average molecular mass of polylactic acid is not limited to the numerical values listed above, and other numerical values unlisted within the numerical range are also applicable. The weight-average molecular mass of the polycaprolactone is 10000 Da to 50000 Da, for example, 10000 Da, 20000 Da, 30000 Da, 40000 Da, or 50000 Da. The weight-average molecular mass of the polycaprolactone is not limited to the numerical values listed above, and other numerical values unlisted within the numerical range are also applicable.

In the present disclosure, by preferably controlling the molecular masses of polylactic acid and polycaprolactone within specific ranges and controlling their concentrations, the coating quality can be further ensured, and the corrosion resistance can be improved. When their molecular masses are excessively high, the solution viscosity increases substantially, and the solution cannot be effectively broken into droplets by ultrasonic vibrations and then clogs the nozzle, thereby causing an elevated equipment failure rate. Furthermore, the excessive molecular masses elevate the glass transition temperature, chain segment mobility during solvent evaporation is restricted, shrinkage stress accumulates, web-like cracks are generated after the coating is dried, and the coating is prone to delamination. At the same time, overlong molecular chains induce excessive chain entanglement, and the coating toughness is reduced, thereby degrading overall coating quality and lowering the corrosion resistance. When the molecular masses are too low, the particle size of ultrasonically atomized droplets becomes excessively small, and the drying rate of these droplets is excessively rapid, thereby resulting in the formation of a porous and friable structure. Due to shortened molecular chains, intermolecular forces weaken significantly, and effective entanglement fails to be formed, thereby compromising the wear resistance of the coating and making the coating susceptible to scratch damage. In addition, low-molecular-mass polylactic acid exhibits higher terminal carboxyl group content (increased hydrolysis initiation sites) and reduced crystallinity, thereby accelerating the degradation rate of polylactic acid and impairing corrosion resistance.

Preferably, a height of an ultrasonic nozzle from the base coating during ultrasonic spraying in step (3) is 10 cm to 25 cm, for example, 10 cm, 12 cm, 14 cm, 16 cm, 18 cm, 20 cm, 22 cm, 24 cm, or 25 cm. The height of the ultrasonic nozzle is not limited to the numerical values listed above, and other numerical values unlisted within the numerical range are also applicable.

In the present disclosure, by preferably controlling the height between the ultrasonic nozzle and the base coating during ultrasonic spraying, the bonding strength and the thickness uniformity of the coating can be controlled. Specifically, when the height is excessive, the kinetic energy of the droplets becomes insufficient, and the droplets are dispersed unevenly, thereby resulting in reduced bonding strength and non-uniform coating thickness. When the height is too low, polylactic acid accumulates, and the solution is spattered, thereby causing thickness non-uniformity, potential coating damage, and reduced corrosion resistance.

A spraying rate of the ultrasonic spraying is 0.08 mL/min to 0.15 mL/min, for example, 0.08 mL/min, 0.10 mL/min, 0.12 mL/min, 0.14 mL/min, or 0.15 mL/min. The spraying rate of the ultrasonic spraying is not limited to the numerical values listed above, and other numerical values unlisted within the numerical range are also applicable.

In the present disclosure, by preferably controlling the spraying rate of the ultrasonic spraying, the atomization efficiency and the interlayer bonding strength can be controlled. Specifically, when the spraying rate is excessively high, the droplets are prone to agglomeration or incomplete atomization, and interlayer spray solutions fail to be fully fused, thereby impairing the interlayer bonding strength. When the spraying rate is too low, the droplets are unevenly distributed due to the energy of ultrasonic, the resulting coating exhibits a non-uniform thickness or even cannot be completely coated, and when subjected to external forces, the coating may be prone to delamination and reduced adhesion. In addition, the too low spraying rate may also cause non-uniform dispersion of nano-hydroxyapatite in the spraying solution and then lead to localized calcification during degradation, thereby compromising the bone healing efficiency.

A gas pressure of the ultrasonic spraying is 0.01 MPa to 0.05 MPa, for example, 0.01 MPa, 0.02 MPa, 0.03 MPa, 0.04 MPa, or 0.05 MPa. The gas pressure of the ultrasonic spraying is not limited to the numerical values listed above, and other numerical values unlisted within the numerical range are also applicable.

In the present disclosure, by preferably controlling the gas pressure of the ultrasonic spraying, deposition efficiency and the structure of the coating can be controlled. Specifically, when the gas pressure is excessively high, the droplets are dispersed by airflow, rapidly evaporated, and are spattered upon substrate impact, and the coating may suffer from orange peel effects or its porosity may increase, thereby compromising the coating quality. When the gas pressure is insufficient, the sprayed liquid mist is uneven, the resulting coating exhibits a non-uniform thickness, and the droplets may accumulate and have a great tendency to sag, thereby causing defects on the coating surface, diminishing process yield and efficiency, and escalating production costs.

Preferably, a power of the ultrasonic spraying in step (3) is 0.5 W to 1.5 W, for example, 0.5 W, 0.6 W, 0.8 W, 1 W, 1.2 W, 1.4 W, or 1.5 W. The power of the ultrasonic spraying is not limited to the numerical values listed above, and other numerical values unlisted within the numerical range are also applicable.

Preferably, a temperature of drying in step (3) is 45° C. to 55° C., for example, 45° C., 46° C., 48° C., 50° C., 52° C., 54° C., or 55° C. The time of drying is not limited to the numerical values listed above, and other numerical values unlisted within the numerical range are also applicable.

In the present disclosure, the primary function of the drying is to volatilize the solvent from the polymer coating, thereby ensuring that the residual solvent remains within safety limits. In the present disclosure, the temperature of the drying is preferably controlled. When the temperature of the drying is excessively high and approaches the glass transition temperature of the polymer material, the coating softens and undergoes secondary curing at room temperature, and this process compromises the bonding strength of the coating. When the temperature of the drying is too low, the volatilization of solvent is incomplete, and the residual solvent leads to product nonconformity.

Preferably, the time of the drying in step (3) is 48 h to 72 h, for example, 48 h, 50 h, 52 h, 54 h, 56 h, 58 h, 60 h, 62 h, 64 h, 66 h, 68 h, or 70 h. The time of the drying is not limited to the numerical values listed above, and other numerical values unlisted within the numerical range are also applicable.

In the present disclosure, the time of the drying is preferably controlled. If the time of the drying is too long, production costs are escalated. If the time of the drying is too short, the solvent cannot be volatilized completely, and the residual solvent is within a non-compliant level.

In the present disclosure, the magnesium alloy substrate may be, for example, any one of an AE series alloy, an AS series alloy, a WE series alloy, an AZ series alloy, an AM series alloy, a ZK series alloy, a ZM series alloy, a Mg—Li series alloy, or a Mg—Ca series alloy and is preferably a series magnesium alloy containing magnesium, zinc, and manganese.

As a preferred technical solution of the second aspect of the present disclosure, the preparation method includes the following steps.
(1) Suspending a magnesium alloy substrate within a reaction vessel containing purified water, sealing the vessel and performing a hydrothermal reaction between water vapor with the magnesium alloy substrate obtain the substrate containing a base coating, where the distance between the magnesium alloy substrate and the purified water is 30 mm to 50 mm, and the hydrothermal reaction is carried out at a temperature of 120° C. to 160° C. and a pressure of 1.0 MPa to 2.0 MPa for 2 h to 6 h.
(2) Immersing the substrate containing the base coating obtained in step (1) in a phosphate buffer solution having a concentration of 8 mmol/L to 15 mmol/L and soaking for 5 min to 15 min at a temperature of 60° C. to 80° C. to obtain the base coating containing microporous channels.
(3) Adding polylactic acid (PLA, with a weight-average molecular mass of 100000 Da to 200000 Da) and polycaprolactone (PCL, with a weight-average molecular mass of 10000 Da to 50000 Da) to dichloromethane, and then magnetically stirring at 25° C. for 1 h to 2 h to fully dissolve polylactic acid and polycaprolactone with no suspended particles observed; adding nano-hydroxyapatite, and continuously magnetically stirring for 15 min to 30 min to evenly disperse nano-hydroxyapatite in the solution to obtain a spraying solution, where in the spraying solution, the concentration of polylactic acid is 0.005 g/mL to 0.020 g/mL, the concentration of polycaprolactone is 0.25 mg/mL to 1.50 mg/mL, and the concentration of nano-hydroxyapatite is 0.05 mg/mL to 0.50 mg/mL.

Ultrasonic spraying is carried out on the base coating obtained in step (2) by using the spraying solution containing a polymer material, and the base coating is dried at 45° C. to 55° C. for 48 h to 72 h to complete the preparation of the magnesium alloy composite coating, where height of the ultrasonic nozzle from the base coating during ultrasonic spraying is 10 cm to 25 cm, the spraying rate is 0.08 mL/min to 0.15 mL/min, and the gas pressure is 0.01 MPa to 0.05 MPa.

In a third aspect, the present disclosure provides a corrosion-resistant medical magnesium alloy for promoting bone healing. The surface of the medical magnesium alloy contains the corrosion-resistant magnesium alloy composite coating for promoting bone healing described in the first aspect of the present disclosure.

The medical magnesium alloy provided by the present disclosure adopts the composite coating, which has good biocompatibility, corrosion resistance, and mechanical properties, can promote bone healing, and is particularly suitable for scenarios requiring high mechanical properties, such as magnesium alloy used for magnesium alloy bone plates and bone nails.

Compared to the related art, the present disclosure has the following beneficial effects.
(1) By adopting the base coating formed by magnesium hydroxide and/or magnesium oxide integrated with the surface coating formed by a polymer material, the composite coating provided by the present disclosure exhibits enhanced corrosion resistance, thereby effectively reducing the degradation rate of the magnesium alloy and expanding the application scope of the magnesium alloy.
(2) In the composite coating provided by the present disclosure, the base coating and the surface coating are bonded to each other in an interdigitation manner, and the composite coating thus achieves superior firmness. When the composite coating is applied to the surface of magnesium alloy bone plates and nails, even under the action of relatively great friction generated during the torsional screw of the bone nails, the composite coating still maintains bonding stability without fracture and exhibits a slow degradation rate, high strength, and exceptional toughness.
(3) In the composite coating and the preparation method therefor provided by the present disclosure, by preferably adding functional components such as nano-hydroxyapatite to the spraying solution, the strength of the surface coating can be improved, and with the continuous release of nano-hydroxyapatite in practice, the adhesion, proliferation, and differentiation of osteocytes can be enhanced, thereby accelerating bone tissue regeneration.
(4) Under optimized conditions, the composite coating provided by the present disclosure can reduce a corrosion current to 70.7 nA or below and elevate a corrosion potential to −1.52 V or above, and the coating retains great structural integrity after three screw-in/screw-out cycles.

### BRIEF DESCRIPTION OF DRAWINGS

FIG. 1 is an SEM image of the base coating containing microporous channels provided by Example 1 of the present disclosure;

FIG. 2 is an SEM image of the composite coating provided by Example 1 of the present disclosure after three screw-in/screw-out cycles;

FIG. 3 is an SEM image of the composite coating provided by Example 4 of the present disclosure after three screw-in/screw-out cycles;

FIG. 4 is an SEM image of the composite coating provided by Comparative Example 1 of the present disclosure after three screw-in/screw-out cycles;

FIG. 5 is a comparison chart of results of corrosion resistance experiments performed on the composite coating provided by Example 1, the coating provided by Comparative Example 3, and the magnesium alloy substrate provided by Comparative Example 4 of the present disclosure;

FIG. 6 is an image showing the bone healing effect of the composite coating provided by Example 1 of the present disclosure for animal experiments; and

FIG. 7 is an image showing the bone healing effect of the composite coating provided by Example 4 of the present disclosure for animal experiments.

### DETAILED DESCRIPTION

Technical solutions of the present disclosure are further described below through specific examples. It is to be understood by those skilled in the art that the examples described below are merely used for a better understanding of the present disclosure and are not to be construed as specific limitations to the present disclosure.

### Example 1

This example provides a corrosion-resistant magnesium alloy composite coating for promoting bone healing. The magnesium alloy composite coating included a base coating and a surface coating which were sequentially arranged outward from the surface of the magnesium alloy.

The base coating included microporous channels, and the component of the base coating included magnesium hydroxide and/or magnesium oxide. The porosity of the base coating was 50%, and the pore diameter of the microporous channels was 20 μm to 120 μm.

The surface coating included a polymer material, and the polymer material included polylactic acid and polycaprolactone. The surface coating filled the microporous channels of the base coating and covered the surface of the base coating. Nano-hydroxyapatite having an average particle size of 30 nm was distributed within the polymer material.

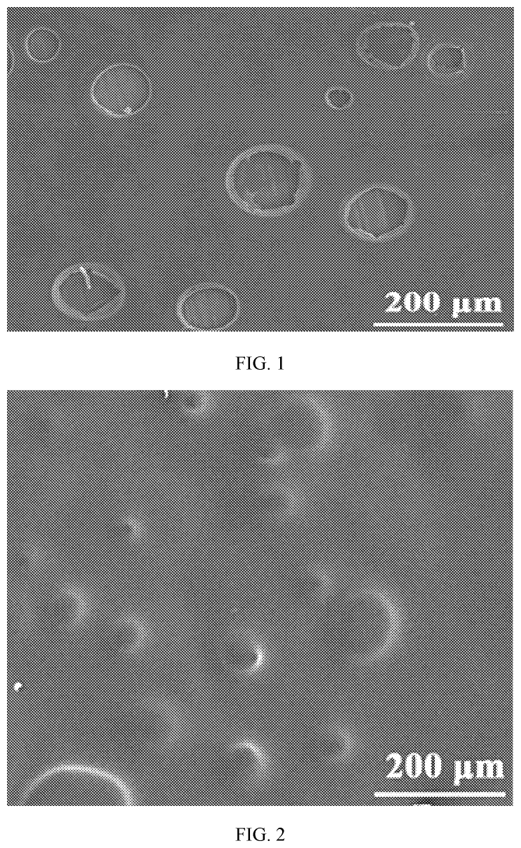

This example provides a method for preparing the above-mentioned magnesium alloy composite coating. The preparation method includes the following steps.
(1) A magnesium alloy substrate (an AE series alloy) was suspended in a reaction vessel containing purified water, where the amount of purified water added was 30% of the volume of the reaction vessel. The vessel was sealed, and a hydrothermal reaction was carried out on the magnesium alloy substrate by using water vapor, where the distance between the magnesium alloy substrate and the purified water was 40 mm, and the hydrothermal reaction was carried out at a temperature of 140° C. and at a pressure of 1.5 MPa for 4 h. After the reaction was completed, the magnesium alloy substrate was naturally cooled and taken out of the reaction vessel, and then the substrate containing a base coating was obtained.
(2) The substrate containing the base coating obtained in step (1) was immersed in a phosphate buffer solution having a concentration of 12 mmol/L and soaked for 10 min at a temperature of 70° C. The substrate was then taken out of the phosphate buffer solution, rinsed with purified water and air-dried, and then the base coating containing microporous channels was obtained, as shown in FIG. 1.
(3) Polylactic acid (PLA, with a weight-average molecular mass of 180000 Da) and polycaprolactone (PCL, with a weight-average molecular mass of 30000 Da) were added to dichloromethane, and then magnetically stirred at 25° C. for 1.5 h to fully dissolve polylactic acid and polycaprolactone with no suspended particles observed. Nano-hydroxyapatite was added, and continuously magnetically stirred for 20 min to evenly disperse nano-hydroxyapatite in the solution to obtain a spraying solution, where in the spraying solution, the concentration of polylactic acid was 0.01 g/mL, the concentration of polycaprolactone was 0.5 mg/mL, and the concentration of nano-hydroxyapatite was 0.2 mg/mL.

Ultrasonic spraying was carried out on the base coating obtained in step (2) by using the spraying solution containing the polymer material, and the base coating was dried at 48° C. for 56 h to complete the preparation of the magnesium alloy composite coating, where the height of the ultrasonic nozzle from the base coating during ultrasonic spraying was 15 cm, the spraying rate was 0.1 mL/min, the gas pressure was 0.02 MPa, the ultrasonic power was 1.2 W, and the thickness of the coating was 20 μm.

This example provides a corrosion-resistant medical magnesium alloy for promoting bone healing. The surface of the medical magnesium alloy contained the above-mentioned corrosion-resistant magnesium alloy composite coating for promoting bone healing.

### Example 2

This example provides a corrosion-resistant magnesium alloy composite coating for promoting bone healing. The magnesium alloy composite coating included a base coating and a surface coating which were sequentially arranged outward from the surface of the magnesium alloy.

The base coating included microporous channels, and the component of the base coating included magnesium hydroxide and/or magnesium oxide. The porosity of the base coating was 55%, and the pore diameter of the microporous channels was 20 μm to 120 μm.

The surface coating included a polymer material, and the polymer material included polylactic acid and polycaprolactone. The surface coating filled the microporous channels of the base coating and covered the surface of the base coating. Nano-hydroxyapatite having an average particle size of 30 nm was distributed within the polymer material.

This example provides a method for preparing the above-mentioned magnesium alloy composite coating. The preparation method includes the following steps.
(1) A magnesium alloy substrate (an AE series alloy) was suspended in a reaction vessel containing purified water, where the amount of purified water added was 30% of the volume of the reaction vessel. The vessel was sealed, and a hydrothermal reaction was carried out on the magnesium alloy substrate by using water vapor, where the distance between the magnesium alloy substrate and the purified water was 40 mm, and the hydrothermal reaction was carried out at a temperature of 140° C. and at a pressure of 1.5 MPa for 4 h. After the reaction was completed, the magnesium alloy substrate was naturally cooled and taken out of the reaction vessel, and then the substrate containing a base coating was obtained.
(2) The substrate containing the base coating obtained in step (1) was immersed in a phosphate buffer solution having a concentration of 8 mmol/L and soaked for 15 min at a temperature of 60° C. The substrate was then taken out of the phosphate buffer solution, rinsed with purified water and air-dried, and then the base coating containing microporous channels was obtained.
(3) Polylactic acid (PLA, with a weight-average molecular mass of 180000 Da) and polycaprolactone (PCL, with a weight-average molecular mass of 30000 Da) were added to dichloromethane, and then magnetically stirred at 25° C. for 1.5 h to fully dissolve polylactic acid and polycaprolactone with no suspended particles observed. Nano-hydroxyapatite was added, and continuously magnetically stirred for 20 min to evenly disperse nano-hydroxyapatite in the solution to obtain a spraying solution, where in the spraying solution, the concentration of polylactic acid was 0.005 g/mL, the concentration of polycaprolactone was 1.5 mg/mL, and the concentration of nano-hydroxyapatite was 0.5 mg/mL.

Ultrasonic spraying was carried out on the base coating obtained in step (2) by using the spraying solution containing the polymer material, and the base coating was dried at 55° C. for 48 h to complete the preparation of the magnesium alloy composite coating, where the height of the ultrasonic nozzle from the base coating during ultrasonic spraying was 10 cm, the spraying rate was 0.15 mL/min, the gas pressure was 0.01 MPa, the ultrasonic power was 1.2 W, and the thickness of the coating was 20 μm.

This example provides a corrosion-resistant medical magnesium alloy for promoting bone healing. The surface of the medical magnesium alloy contained the above-mentioned corrosion-resistant magnesium alloy composite coating for promoting bone healing.

### Example 3

This example provides a corrosion-resistant magnesium alloy composite coating for promoting bone healing. The magnesium alloy composite coating included a base coating and a surface coating which were sequentially arranged outward from the surface of the magnesium alloy.

The base coating included microporous channels, and the component of the base coating included magnesium hydroxide and/or magnesium oxide. The porosity of the base coating was 40%, and the pore diameter of the microporous channels was 20 μm to 120 μm.

The surface coating included a polymer material, and the polymer material included polylactic acid and polycaprolactone. The surface coating filled the microporous channels of the base coating and covered the surface of the base coating. Nano-hydroxyapatite having an average particle size of 30 nm was distributed within the polymer material.

This example provides a method for preparing the above-mentioned magnesium alloy composite coating. The preparation method includes the following steps.
(1) A magnesium alloy substrate (an AE series alloy) was suspended in a reaction vessel containing purified water, where the amount of purified water added was 30% of the volume of the reaction vessel. The vessel was sealed, and a hydrothermal reaction was carried out on the magnesium alloy substrate by using water vapor, where the distance between the magnesium alloy substrate and the purified water was 40 mm, and the hydrothermal reaction was carried out at a temperature of 140° C. and at a pressure of 1.5 MPa for 4 h. After the reaction was completed, the magnesium alloy substrate was naturally cooled and taken out of the reaction vessel, and then the substrate containing a base coating was obtained.
(2) The substrate containing the base coating obtained in step (1) was immersed in a phosphate buffer solution having a concentration of 15 mmol/L and soaked for 5 min at a temperature of 80° C. The substrate was then taken out of the phosphate buffer solution, rinsed with purified water and air-dried, and then the base coating containing microporous channels was obtained.
(3) Polylactic acid (PLA, with a weight-average molecular mass of 180000 Da) and polycaprolactone (PCL, with a weight-average molecular mass of 30000 Da) were added to dichloromethane, and then magnetically stirred at 25° C. for 1.5 h to fully dissolve polylactic acid and polycaprolactone with no suspended particles observed. Nano-hydroxyapatite was added, and continuously magnetically stirred for 20 min to evenly disperse nano-hydroxyapatite in the solution to obtain a spraying solution, where in the spraying solution, the concentration of polylactic acid was 0.02 g/mL, the concentration of polycaprolactone was 0.25 mg/mL, and the concentration of nano-hydroxyapatite was 0.05 mg/mL.

Ultrasonic spraying was carried out on the base coating obtained in step (2) by using the spraying solution containing the polymer material, and the base coating was dried at 45° C. for 72 h to complete the preparation of the magnesium alloy composite coating, where the height of the ultrasonic nozzle from the base coating during ultrasonic spraying was 25 cm, the spraying rate was 0.08 mL/min, the gas pressure was 0.05 MPa, the ultrasonic power was 1.2 W, and the thickness of the coating was 20 μm.

This example provides a corrosion-resistant medical magnesium alloy for promoting bone healing. The surface of the medical magnesium alloy contained the above-mentioned corrosion-resistant magnesium alloy composite coating for promoting bone healing.

### Example 4

This example provides a corrosion-resistant magnesium alloy composite coating for promoting bone healing. This example differs from Example 1 only in that nano-hydroxyapatite was not added to the surface coating.

This example further provides a method for preparing the above-mentioned magnesium alloy composite coating. This example differs from Example 1 only in that nano-hydroxyapatite was not added to the spraying solution in step (3).

### Example 5

This example provides a method for preparing a corrosion-resistant magnesium alloy composite coating for promoting bone healing. This example differs from Example 1 only in that the soaking in step (2) was carried out at a temperature of 55° C.

### Example 6

This example provides a method for preparing a corrosion-resistant magnesium alloy composite coating for promoting bone healing. This example differs from Example 1 only in that the soaking in step (2) was carried out at a temperature of 85° C.

### Example 7

This example provides a method for preparing a corrosion-resistant magnesium alloy composite coating for promoting bone healing. This example differs from Example 1 only in that the soaking in step (2) was carried out for 2 min.

### Example 8

This example provides a method for preparing a corrosion-resistant magnesium alloy composite coating for promoting bone healing. This example differs from Example 1 only in that the soaking in step (2) was carried out for 20 min.

### Example 9

This example provides a method for preparing a corrosion-resistant magnesium alloy composite coating for promoting bone healing. This example differs from Example 1 only in that the spraying rate during ultrasonic spraying in step (3) was 0.05 mL/min.

### Example 10

This example provides a method for preparing a corrosion-resistant magnesium alloy composite coating for promoting bone healing. This example differs from Example 1 only in that the spraying rate during ultrasonic spraying in step (3) was 0.18 mL/min.

### Example 11

This example provides a method for preparing a corrosion-resistant magnesium alloy composite coating for promoting bone healing. This example differs from Example 1 only in that the gas pressure during ultrasonic spraying in step (3) was 0.005 MPa.

### Example 12

This example provides a method for preparing a corrosion-resistant magnesium alloy composite coating for promoting bone healing. This example differs from Example 1 only in that the gas pressure during ultrasonic spraying in step (3) was 0.08 MPa.

### Comparative Example 1

This comparative example provides a method for preparing a magnesium alloy coating. This comparative example differs from Example 1 only in that steps (1) and (2) were not carried out and the surface of the magnesium alloy substrate was directly sprayed by using the method in step (3).

### Comparative Example 2

This comparative example provides a method for preparing a magnesium alloy composite coating. This comparative example differs from Example 1 only in that step (2) was not carried out and the substrate containing the base coating obtained in step (1) was directly sprayed by using the method in step (3).

### Comparative Example 3

This comparative example provides a method for preparing a magnesium alloy coating. This comparative example differs from Example 1 only in that steps (2) and (3) were not carried out and the magnesium alloy coating was only a base coating.

### Comparative Example 4

This comparative example provides a magnesium alloy substrate. The magnesium alloy substrate was the same as the magnesium alloy substrate of Example 1.

### Comparative Example 5

This comparative example provides a method for preparing a magnesium alloy composite coating. This comparative example differs from Example 1 only in that step (3) was replaced by: equal amounts of polylactic acid, polycaprolactone, and nano-hydroxyapatite were mixed and molded at 200° C. and 50 N to form a polymer flake, and the polymer flake was placed above the base coating containing microporous channels and hot-pressed at 200° C. and 20 MPa to obtain a composite coating.

The products obtained from the above examples and comparative examples were examined for coating integrity after being subjected to three screw-in/screw-out cycles of a Grade 40 polyurethane material (simulating cortical bone hardness).

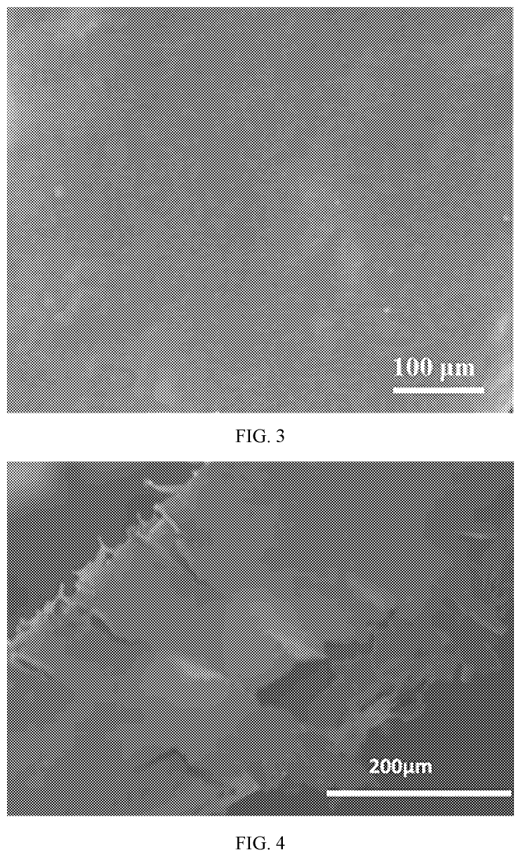

Taking the products provided by Example 1, Example 4, and Comparative Example 1 as examples, the SEM images of these three products after three screw-in/screw-out cycles are shown in FIG. 2, FIG. 3, and FIG. 4, respectively. As can be seen from FIG. 2, the composite coating provided by Example 1 exhibited complete surface integrity, and particulate nano-hydroxyapatite was distributed within the surface coating. As can be seen from FIG. 3, the composite coating provided by Example 4 still maintained good integrity, and the coating had a good bonding strength to the magnesium alloy substrate; the energy-dispersive X-ray spectroscopy (EDS) analysis revealed that the primary elements in the coating were C and O, which were the main component elements of polylactic acid, indicating that the polymer coating was effectively bonded to the surface; the coating thickness was measured using a film thickness gauge, and the measured thickness of the surface coating was 16.5 μm. As can be seen from FIG. 4, after three screw-in/screw-out cycles, the polymer coating provided by Comparative Example 1 showed cracking and was delaminated from the magnesium alloy substrate.

The corrosion resistance of the coatings in their service periods was evaluated using an electrochemical workstation after the coatings were subjected to three screw-in/screw-out cycles. The electrochemical tests employed a PBS solution having a pH of 7.4 and were carried out at an initial potential of −0.3 V and a final potential of 0.3 V for 120 s, with a scan rate of 1 mV/s. The results are listed in Table 1.

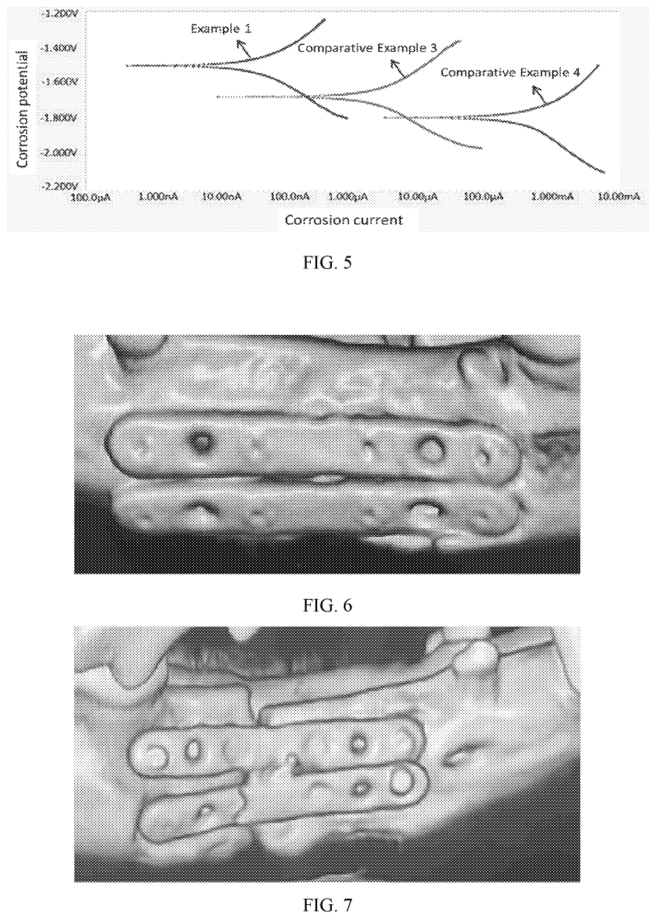

Taking the comparison of the magnesium alloy with the composite coating provided by Example 1, the magnesium alloy with only the base coating provided by Comparative Example 3, and the bare magnesium alloy provided by Comparative Example 4 as an example, the samples having the same shape after three screw-in/screw-out cycles were subjected to tests for corrosion-resistance performance by an electrochemical workstation, and the comparison results are shown in FIG. 5. As shown in FIG. 5, the composite coating exhibited superior corrosion resistance compared to both the base coating and the bare magnesium alloy substrate, demonstrating a significant enhancement in corrosion resistance.

Taking Example 1 and Example 4 as examples, the resulting magnesium alloy products were applied in animal experiments. The experiments were carried out in the following method: gingival tissue was separated from the mandible of a dog, and a fracture model was created at an appropriate site using a surgical instrument; after the fracture site was repeatedly rinsed with saline solution to remove bone debris, medical magnesium alloys coated with the composite coating provided by the above examples were fabricated into bone plate and nail systems and then implanted for mandibular fracture repair and fixation, with two sets of the plate-nail systems implanted unilaterally in parallel. The bone healing effects in the animal experiments were shown in FIGS. 6 and 7, respectively. As can be seen from FIG. 6, the magnesium alloy product provided by Example 1 demonstrated accelerated bone healing efficiency, the fracture line healed in time, and the bone plates and the healing of the fracture line achieved good temporal compatibility. As can be seen from FIG. 7, the magnesium alloy product provided by Example 4 exhibited slower bone healing efficiency, the fracture line failed to be healed promptly, and the bone plates were subjected to stress, resulting in stress-induced corrosion and the fracture of the bone plates.

TABLE 1

| | | | Coating |
|---|---|---|---|
| | Corrosion | Corrosion | integrity after |
| | current/nA | potential/V | screw-in/screw-out |
| Example 1 | 55.1 | −1.50 | Yes |
| Example 2 | 65.3 | −1.51 | Yes |
| Example 3 | 70.7 | −1.52 | Yes |
| Example 4 | 68.8 | −1.51 | Yes |
| Example 5 | 1573.8 | −1.72 | No |
| Example 6 | 2161.1 | −1.75 | No |
| Example 7 | 1753.1 | −1.73 | No |
| Example 8 | 2574.5 | −1.76 | No |
| Example 9 | 1577.9 | −1.73 | No |
| Example 10 | 2578.3 | −1.76 | No |
| Example 11 | 2199.9 | −1.75 | No |
| Example 12 | 2388.7 | −1.75 | No |
| Comparative | 8543.9 | −1.80 | No |
| Example 1 | | | |
| Comparative | 3715.2 | −1.78 | No |
| Example 2 | | | |
| Comparative | 4674.6 | −1.77 | Yes |
| Example 3 | | | |
| Comparative | 786531.9 | −1.81 | — |
| Example 4 | | | |
| Comparative | 15876.7 | −1.81 | No |
| Example 5 | | | |

“-” in Table 1 indicates the absence of a coating.

As can be seen from the data in Table 1:
(1) As can be seen from the data of Examples 1 to 4, under optimized conditions, by employing the composite coating provided by the present disclosure, the corrosion current can be reduced to 70.7 nA or below, the corrosion potential can be elevated to −1.52 V or above, and the coating retains great structural integrity after three screw-in/screw-out cycles.
(2) As can be seen from the data of Example 1 and Examples 5 to 8, in the present disclosure, by preferably controlling the temperature and time of the soaking in step (2), the structure of the microporous channels and the surface roughness of the base coating can be better controlled, thereby improving the bonding strength of the coating and enhancing the corrosion resistance and mechanical properties of the composite coating.
(3) As can be seen from the data of Example 1 and Examples 9 to 12, in the present disclosure, by preferably controlling the parameters of ultrasonic spraying, such as the spraying rate and the gas pressure, the quality of the surface coating can be further improved, thereby enhancing the corrosion resistance and mechanical properties of the composite coating.
(4) As can be seen from the data of Example 1 and Comparative Examples 1 to 5, compared to the bare magnesium alloy provided by Comparative Example 4, the composite coating provided by Example 1 can effectively enhance corrosion resistance, thereby inhibiting the degradation of the magnesium alloy; compared to the magnesium alloy having only the surface coating in Comparative Example 1 and the magnesium alloy having only the base coating in Comparative Example 3, the magnesium alloy in Example 1 exhibits superior corrosion resistance and higher coating integrity; compared to the magnesium alloy composite coating which lacks microporous channels to form an interdigitation connection in Comparative Example 2, the coating in Example 1 demonstrates a higher bonding strength, maintains higher integrity, and provides better corrosion resistance; compared to the magnesium alloy composite coating where the base coating and the surface coating are bonded via hot pressing in Comparative Example 5, the coating in Example 1 shows improved corrosion resistance and mechanical properties. In Comparative Example 5, the coating suffers from poor coating firmness, and furthermore, since the hot-pressing temperature significantly exceeds the glass transition temperature of polylactic acid, which results in polymer chain scission and reduced molecular mass, the coating has inferior corrosion resistance.

In summary, by bonding the base coating formed by a chemical conversion method to the surface coating formed by a polymer material in an interdigitation manner, the magnesium alloy composite coating provided by the present disclosure has good firmness, strength and toughness, improves the corrosion resistance of the magnesium alloy, and meets the need of promoting bone healing.

The applicant states that the above are the specific embodiments of the present disclosure and are not intended to limit the protection scope of the present disclosure. Those skilled in the art should understand that any changes or substitutions easily conceivable by those skilled in the art within the technical scope disclosed in the present disclosure fall within the protection scope and the disclosed scope of the present disclosure.

What is claimed is:

1. A corrosion-resistant magnesium alloy composite coating for promoting bone healing, comprising a base coating and a surface coating which are sequentially arranged outward from a surface of a magnesium alloy;
wherein the base coating comprises microporous channels, and a component of the base coating comprises magnesium hydroxide and/or magnesium oxide; the base coating containing the microporous channels is obtained by immersing and soaking a substrate containing the base coating in a phosphate buffer solution, and the soaking is carried out at a temperature of 60° C. to 80° C. for 5 min to 15 min, and a porosity of the base coating is 30% to 60%;
the surface coating comprises a polymer material, the polymer material comprises polylactic acid or a mixture of polylactic acid and polycaprolactone, nano-hydroxyapatite is distributed within the polymer material, and the surface coating fills the microporous channels of the base coating and covers a surface of the base coating.

2. The magnesium alloy composite coating according to claim 1, wherein a particle size of the nano-hydroxyapatite is 20 nm to 60 nm.

3. The magnesium alloy composite coating according to claim 1, wherein a pore diameter of the microporous channels is 20 μm to 120 μm.

4. A method for preparing the corrosion-resistant magnesium alloy composite coating for promoting bone healing according to claim 1, comprising the following steps:
(1) carrying out a hydrothermal reaction on a magnesium alloy substrate by using water vapor to obtain a substrate containing a base coating;
(2) immersing and soaking the substrate containing the base coating obtained in step (1) in a phosphate buffer solution to obtain a base coating containing microporous channels, wherein the soaking is carried out at a temperature of 60° C. to 80° C. for 5 min to 15 min; and
(3) carrying out ultrasonic spraying on the base coating obtained in step (2) by using a spraying solution containing a polymer material, and drying the base coating to complete the preparation of the magnesium alloy composite coating.

5. The preparation method according to claim 4, wherein a concentration of the phosphate buffer solution in step (2) is 8 mmol/L to 15 mmol/L.

6. The preparation method according to claim 4, wherein a concentration of polylactic acid in the spraying solution in step (3) is 0.005 g/mL to 0.020 g/mL;
a concentration of polycaprolactone in the spraying solution is 0.25 mg/mL to 1.50 mg/mL.

7. The preparation method according to claim 6, wherein a concentration of nano-hydroxyapatite in the spraying solution in step (3) is 0.05 mg/mL to 0.50 mg/mL.

8. The preparation method according to claim 6, wherein a weight-average molecular mass of the polylactic acid in step (3) is 100000 Da to 200000 Da;
a weight-average molecular mass of the polycaprolactone is 10000 Da to 50000 Da.

9. The preparation method according to claim 4, wherein a height of an ultrasonic nozzle from the base coating during the ultrasonic spraying in step (3) is 10 cm to 25 cm;
a spraying rate of the ultrasonic spraying is 0.08 mL/min to 0.15 mL/min;
a gas pressure of the ultrasonic spraying is 0.01 MPa to 0.05 MPa.

10. A corrosion-resistant medical magnesium alloy for promoting bone healing, and a surface of the medical magnesium alloy contains the corrosion-resistant magnesium alloy composite coating for promoting bone healing according to claim 1.
